(19) United States
(12) Patent Application Publication
(10) Pub. No.: US 2026/0250620 A1
(43) Pub. Date: Aug. 27, 2026

(54) SYSTEM FOR SIMULTANEOUS CONTRACTILE FORCE AND CALCIUM/VOLTAGE TRANSIENT MEASUREMENT OF ENGINEERED TISSUE

(71) Applicant: THE JOHNS HOPKINS UNIVERSITY, Baltimore, MD (US)

(72) Inventors: Deok-Ho KIM, Seattle, WA (US); Amir MONEMIANESFAHANI, Baltimore, MD (US); Patarajarin AKARAPIPAD, Baltimore, MD (US); Byunggik KIM, Baltimore, MD (US)

(21) Appl. No.: 18/994,152

(22) PCT Filed: Jul. 25, 2023
(86) PCT No.: PCT/US2023/028595

§ 371 Date: Jan. 14, 2025

Related U.S. Application Data
(60) Provisional application No. 63/392,013, filed on Jul. 25, 2022.

(51) Int. Cl. C12M 1/34; C12M 1/32; C12M 1/36; C12M 1/42; C12N 5/00

(52) U.S. Cl. CPC C12M 41/46; C12M 23/12; C12M 35/02; C12M 35/04; C12M 35/06; C12M 41/38; C12M 41/48; C12N 5/0062

(57) ABSTRACT

Devices and techniques for a system for simultaneous contractile force and calcium/voltage transient measurement of engineered tissue are generally described. The system for simultaneous contractile force and calcium/voltage transient measurement of engineered tissue of the present disclosure comprises multiple arrays of magnetic sensors and LEDs, an optical unit, and a housing module. This system can simultaneously provide contractile force measurement and calcium/voltage transient observation. It can be integrated with the medium-to high-throughput tissue culture platforms. The system for simultaneous contractile force and calcium/voltage transient measurement of engineered tissue can be utilized in various applications, including but not limited to monitoring the maturation of the tissue, observing muscle conditions, and disease modeling.

### CROSS-REFERENCE TO RELATED APPLICATIONS

This application is the national stage entry of International Patent Application No. PCT/US2023/028595, filed on Jul. 25, 2023, and published as WO 2024/025891 A1 on Feb. 1, 2024, which claims the benefit of U.S. Provisional Patent Application No. 63/392,013, filed on Jul. 25, 2022, which are hereby incorporated by reference in their entireties.

### GOVERNMENT SUPPORT

This invention was made with government support under grant TR003271 awarded by the National Institutes of Health. The government has certain rights in the invention.

### FIELD

Implementations of the disclosure relate generally to tissue engineering and, more particularly, a system that allows the simultaneous measurement of the contractile force and calcium/voltage transient of the engineered tissue.

### BACKGROUND

The continuous improvement in engineered tissue technology has provided us with promising solutions for improving the quality of human life, especially in regenerative medicine, disease modeling, and drug development. To closely mimic the native microphysiological environments and enhance the maturation of engineered tissues, many research groups have applied different types of stimulations to the 3-dimensional (3D) cultured tissues, e.g., electrical stimulation, chemical stimulation, and mechanical stimulation to induce myotube formation and enhance contractile function. Furthermore, these stimulations can also be used to study the unique functions of specific tissue such as using electrical stimulation to study muscle contraction. Nevertheless, either by providing the stimulations for maturation or studying the functions of the engineered tissues, one of the important keys is the ability to monitor the states or responses of the engineered tissues in real time. Sensors can be applied to the system to continuously monitor the physiological responses of the tissues. The derived data can be analyzed and used for precisely adjusting the appropriate level of stimulation or studying the effects or responses when different conditions are applied to the tissue model.

For example, many research groups have utilized 3D-engineered tissue models to study muscular systems since the applications of engineering techniques allow the highly active and functional features of muscles to be precisely studied and observed. Muscle tissue is considered more than 40% of the human body and is responsible for various functions such as facilitating movement and controlling the circulatory system. Muscle force generation is the result of the contraction of stimulated muscle cells (myocytes) that contain myofibrils, composed of thick and thin filaments that slide past each other, contracting the cell shape and producing tensile force. Calcium ion plays an important role in muscle contraction regulation. When a myocyte is stimulated, calcium ions stored in the sarcoplasmic reticulum are released to bind to troponin, causing a conformational change and enabling a cross-bridge muscle contraction cycle.

The study of muscle contraction force and calcium activity in engineered muscle tissue may allow researchers and physicians to assess muscle maturity and health status, develop disease models, study regenerative medicine, and test potential compounds for drug discovery. For instance, in Duchenne Muscular Dystrophy disease, studies have shown that the calcium activity in skeletal muscle may be associated with the resulting irregular contraction force. So, the simultaneous measurement and assessment of calcium-related contraction activities combined with contractile force measurement allow us to better understand the disease physiology and function and study for proper treatments with non-invasive methods.

Researchers have applied engineering innovation to improve 3D-engineered muscle tissues and enable functional muscle tissue contraction in a controllable and observable manner. One of the most common techniques that allow the assessment of the muscle contractile function is the 2-post system in which a tissue is formed around 2 posts (one is rigid and another one is flexible). To enable medium and high throughput studies, our platform utilizes a magnetic sensor to detect the movement of a small magnet embedded in the tip of the flexible post when muscle tissue contracts. For the calcium transient measurement, a calcium-sensitive dye is used to track calcium activity in muscle tissues. For the voltage transient measurement, a voltage-sensitive dye is used to track voltage activity in muscle tissues. The appropriate excitation light source together with an optical filter is required to view the fluorescence signal.

### SUMMARY

In accordance with examples of the present disclosure, a simultaneous contractile force and calcium/voltage measurement system is disclosed. The system comprises multiple magnetic sensors and LEDs arrays mounted on a PCB for monitoring tissue conditions; an optical unit for observing tissue and calcium activity; a housing module for controlling temperature and enabling 3D movement of the base or camera.

In accordance with examples of the present disclosure, a method is disclosed that comprises culturing muscle tissue, neuromuscular tissue, vascularized cardiac tissue, or skeletal muscle tissue around the previously described 2-post system, wherein the flexible post contains a magnetic portion and another post is rigid; providing stimulation such as electrical stimulation to the tissue for observing contractile function in the housing module with appropriate temperature-controlled; detecting a change in a magnetic field as a result of a deflection of the flexible post and determining the force from the filtered signal; detecting the calcium transient by using calcium-sensitive dye or detecting voltage transient by using a voltage-sensitive dye, providing the appropriate excitation light, and monitoring the fluorescence signal via an optical unit.

Various additional features can be included in the system and the method including one or more of the following features. The magnetometer comprises a sensor such as a unipolar ratiometric hall effect sensor. The magnetic sensor unit comprises a capacitor located between the voltage supply pin and ground pin, and the output signal is connected to a low pass filter with the cutoff frequency above, for example, 21 Hz. Together comprising a resistor and capacitor for noise reduction. The hall effect sensor produces increasing voltage signal output that corresponds to the detected increase in magnetic field strength, enable the position sensing. The optical unit comprises an LED mounted on the PCB with a resistor and connected to the transistor for controlling via a microcontroller or data acquisition device with an appropriate wavelength for providing light to excite calcium dye or voltage dye embedded in the tissue. The light filter film with the wavelength cutoff between the required excitation wavelength and emission wavelength and light diffusor sheet are stacked on top of the LEDs to improve the quality and uniformity of the overall excitation light. The camera is mounted to a motorized stage, and an appropriate optical emission filter and lens are applied to monitor tissue activity. The magnetic sensor and LED circuits may be arranged together along with the other circuitry such as for filtering/and or amplifying on the PCB according to the location of the tissue. The housing module comprises a 3D motorized device such as a modified 3D printer to precisely control camera movement for adjusting focus and control the temperature and an enclosure to control the ambient light. The camera is mounted to the 2-dimensional (2D) motorized stage of the modified 3D printer by using the designed 3D printed holder, and the PCB is localized in the designed 3D printed container which allows the tissue plate to fit above closely to maximize magnetic sensor sensitivity; the container is placed on the bed of the modified 3D printer and underneath the camera for tissue conditions observation.

In accordance with examples of the present disclosure, a simultaneous contractile force, calcium, or voltage measurement system is disclosed. The system comprises multiple magnetic position sensors and light emitting diodes (“LEDs”) or Laser Diodes (“LDs”) arrays for monitoring tissue conditions; an optical unit for observing tissue activity, calcium activity, voltage activity, or combinations thereof; and a housing module for controlling temperature and carbon dioxide environment enabling 3D movement of the base or camera.

Various additional features can be included in the system including one or more of the following features. The simultaneous contractile force, calcium, or voltage measurement system can include a magnetometer that comprises analog or digital type multi-axis magnetic position sensor. The multiple magnetic position sensors and light emitting diodes (“LEDs”) or Laser Diodes (“LDs”) arrays are mounted on a printed circuit board (“PCB”). Each of the magnetic position sensors comprise a capacitor located between a voltage supply pin and a ground pin on the PCB and an output signal is connected to a bandpass filter. The bandpass filter is implemented at a cutoff frequency of 21 Hz or above. The bandpass filter can be configured to filter the noise associated with the magnetic sensing using a low pass filtering in the circuit. The magnetic position sensor is effective in increasing in voltage signal due to an increase detected in a magnetic field. The LEDs or LDs are configured with an appropriate wavelength for providing light to excite calcium dye. The system further comprises a light filter film with the wavelength cutoff between the required excitation wavelength and emission wavelength and light diffusor sheet are stacked on top of the LEDs to improve the quality and uniformity of the overall excitation light. The system can further comprise a camera that is mounted to a motorized stage, and an appropriate optical emission filter and lens are applied to monitor tissue activity. The magnetic position sensor and LED or LD circuits may be arranged together along with auxiliary circuitry that sync, filters, amplifies, or both filters and amplifies the output signal on the PCB according to a location of the tissue. The system can further comprise a housing module that comprises a multi motorized axis robot to precisely control camera movement for adjusting focus and to control the temperature and an enclosure to control the ambient light. The multi-motorized axis robot comprises a computer-controlled actuation system. The camera is mounted to one of the 2D surfaces comprised of two available axes of the robot, and the PCB is localized in the auxiliary container which allows the tissue plate to fit above and within the effective working range of magnetic position sensors; the container may be placed in between of the camera and auxiliary container for tissue conditions observation. The tissue is muscle tissue, neuromuscular tissue, vascularized cardiac tissue, or skeletal muscle tissue. The system further comprises a stimulation plate that comprises a plurality of wells. The stimulation plate comprises 24 wells, 40 wells, or 96 wells.

In accordance with examples of the present disclosure, a method is disclosed that comprises culturing muscle tissue, neuromuscular tissue, vascularized cardiac tissue, or skeletal muscle tissue around a 2-post system, wherein the 2-post system comprises a flexible post that contains a magnetic portion and a rigid post; providing stimulation such as electrical stimulation to the tissue for observing contractile function in the housing module with appropriate temperature-controlled; detecting a change in a magnetic field as a result of a deflection of the flexible post and determining the force from the filtered signal; detecting the calcium transient by using calcium-sensitive dye or detecting voltage transient by using voltage-sensitive dye; providing the appropriate excitation light; and monitoring the fluorescence signal via an optical unit. The stimulation can comprise electrical stimulation, alternative approach may comprise mechanical actuation and optogenetic stimulation of the biological sample.

### BRIEF DESCRIPTION OF THE FIGURES

FIG. 1 is an overall illustration of the system for simultaneous contractile force and calcium/voltage transient measurement of engineered tissue setup.

FIG. 2 is a schematic of the PCB containing magnetic sensors, LEDs, and a circuit diagram.

FIG. 3 is the optical monitoring unit.

FIG. 4 is a workflow schematic of a single unit of the two-post system that is monitored using the system for simultaneous contractile force and calcium/voltage transient measurement of engineered tissue.

FIG. 5 is the illustration of simultaneous measurement of the post deflection and calcium activity.

FIG. 6 is the simultaneous contractile force-calcium transient measurement preliminary data.

### DETAILED DESCRIPTION

Reference will now be made in detail to the present embodiments, examples of which are illustrated in the accompanying drawings. Wherever possible, the same reference numbers will be used throughout the drawings to refer to the same or like parts.

Notwithstanding that the numerical ranges and parameters setting forth the broad scope of the embodiments are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less than 10” can assume negative values, e.g., −1, −2, −3, −10, −20, −30, etc.

The following embodiments are described for illustrative purposes only with reference to the figures. Those of skill in the art will appreciate that the following description is exemplary in nature and that various modifications to the parameters set forth herein could be made without departing from the scope of the present embodiments. It is intended that the specification and examples be considered as examples only. The various embodiments are not necessarily mutually exclusive, as some embodiments can be combined with one or more other embodiments to form new embodiments. It will be understood that the structures depicted in the figures may include additional features not depicted for simplicity, while depicted structures may be removed or modified.

### Technical Problem

Some research groups have studied muscle contraction or calcium/voltage transient using entirely optical or video-based measurements for monitoring the post deflection or fluorescence signal. Nevertheless, these studies can only achieve low throughput monitoring and need further post-processing. To our knowledge, simultaneously studying calcium/voltage activity with muscle tissue, neuromuscular tissue, vascularized cardiac tissue, or skeletal muscle tissue contraction has never been properly conducted on a high-throughput platform using magnetic sensors to measure force integrated with optical detection to track calcium/voltage transition.

### Technical Solution

Therefore, in this study, we have integrated the medium-to high-throughput magnetic detection of muscle tissue, neuromuscular tissue, vascularized cardiac tissue, or skeletal muscle tissue contractile force and calcium/voltage transient measurement utilizing the previously established platform. The first goal is to design the array of magnetic sensors integrated with the array of LEDs on the printed circuit board (PCB) to detect muscle contractile force and excite the calcium-sensitive dye for calcium transient measurement or excite the voltage-sensitive dye for voltage transient measurement. The second goal is to design the setup for monitoring calcium/voltage transient signal, facilitating optical focusing, and providing appropriate temperature for maintaining a suitable physiological environment during muscle contraction.

### Advantageous Effects

The present disclosure relates to the system for simultaneous contractile force and calcium/voltage transient measurement of engineered tissue, which is comprised of a PCB containing arrays of magnetic sensors and LEDs, an optical unit, plus a housing module for monitoring the tissue functions and controlling the environment. The arrays of magnetic sensors and LEDs can be arranged in various configurations based on the location of each set of the two-post system such as in medium- or high-throughput well plate formats. A magnetic sensor such as a Hall effect sensor can detect the change in the magnetic field due to the movement of the magnetic material embedded post upon the tissue contraction. Simultaneously, the fluorescence signal from the calcium-sensitive dye or the voltage-sensitive dye can also be monitored through the optical unit, comprised of a camera, optical filter, and lens. The housing module contains a motorized 3D movement control, box enclosure, and temperature and humidity control to provide a suitable environment during the measurement.

The other aspects and advantages of the invention will be presented in the following description. References are made to accompanying drawings which form a part hereof, and in which they are shown by a preferred embodiment of the invention. However, such illustrations do not necessarily represent the full scope of the invention. Therefore, the references are made to the claims and for interpreting the scope of the invention.

In the following description, reference is made to the accompanying drawings that demonstrate embodiments of the present disclosure. It is to be understood that other embodiments may be used, and changes in systems or methods may be made without leaving the scope of the present disclosure. The following description is not to be taken in a limiting sense, and the extent of the embodiments of the present invention is defined only by the claims of the issued patent. It is to be understood that drawings are not necessarily drawn to scale.

Devices and techniques for a system for simultaneous contractile force and calcium/voltage transient measurement of engineered tissue are generally described. The system for simultaneous contractile force and calcium/voltage transient measurement of engineered tissue of the present disclosure comprises multiple arrays of magnetic sensors and LEDs, an optical unit, and a housing module. This system can simultaneously provide contractile force measurement and calcium/voltage transient observation. It can be integrated with the medium-to high-throughput tissue culture platforms. The system for simultaneous contractile force and calcium/voltage transient measurement of engineered tissue can be utilized in various applications, including but not limited to monitoring the maturation of the tissue, observing muscle conditions, and disease modeling.

Several embodiments of the present disclosure provide improved systems and methods for contractile force and calcium/voltage transient measurement of the tissue under a controlled environment. The composition of the simultaneous contractile force and calcium/voltage measurement system includes at least a magnetic sensor and an LED to facilitate the monitoring of tissue conditions.

In the above composition, the magnetic sensor may be but is not limited to, DRV5056 Unipolar Ratiometric Linear Hall Effect Sensor. In some examples in which the magnetic sensors are DRV5056, the magnetic sensor circuit 204 may be designed as follows. A 0.1 μF capacitor may be placed close to the sensor that connects the supply voltage 208 to the ground, and the output voltage from the magnetic sensor may connect to a low pass filter composed of, for instance, a 10 kOhm resistor and a 4.7 μF capacitor, which is connected to ground. To compensate for interfering noise in the magnetic sensor output signal, additional magnetic sensor circuits can be installed on the PCB in the area that is extended beyond the tissue culture plate region to acquire any ambient signal to normalize with the signal received from the sensors intended for each tissue. The voltage output signal can be connected to the data acquisition device 408 for further analysis.

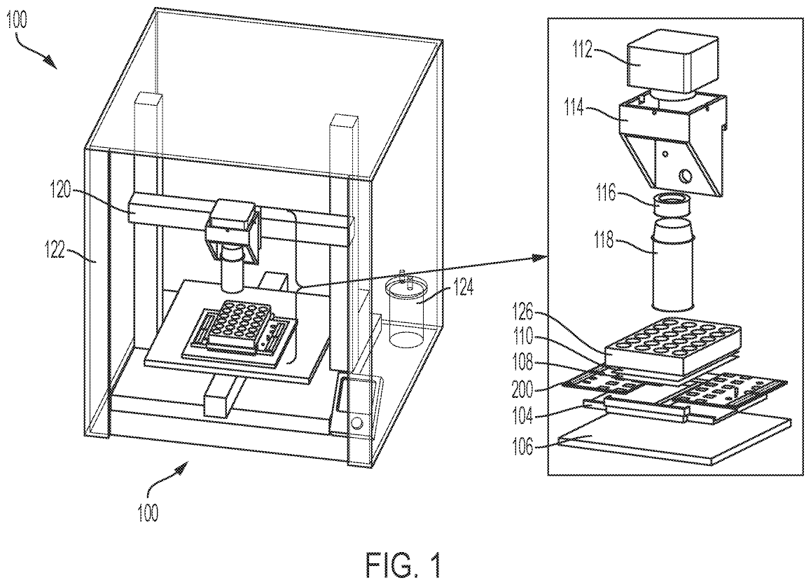

FIG. 1 is an overall illustration of the system for simultaneous contractile force and calcium/voltage transient measurement of engineered tissue setup. In some examples, to calibrate the voltage output signal of the magnetic sensor to the displacement of the deflected post, the flexible post can be manually deflected to mimic the muscle contraction scenario, and the optical camera can be used to monitor the post deflection, while simultaneously the magnetic sensor collects the signal output of the change in the magnetic field. The derived voltage data can be correlated to the displacement and translated to force using a previously described mathematical model. In the above composition, a LED may provide the light source at the wavelength of about 30 nm around the suggested optimal peak excitation wavelength of the calcium-sensitive dye or the voltage-sensitive dye used with the tissue. For example, if the calcium-sensitive dye has an optical peak excitation wavelength of around 490 nm, then an LED with a peak wavelength between 450 nm to 470 nm can be used. Also, if the peak emission wavelength of the dye is 515 nm, the optical filter 116 with the bandwidth cut-on between the excitation wavelength and the emission wavelength can be used, such as the FITC emission filter with the emission wavelength between 513 nm and 556 nm.

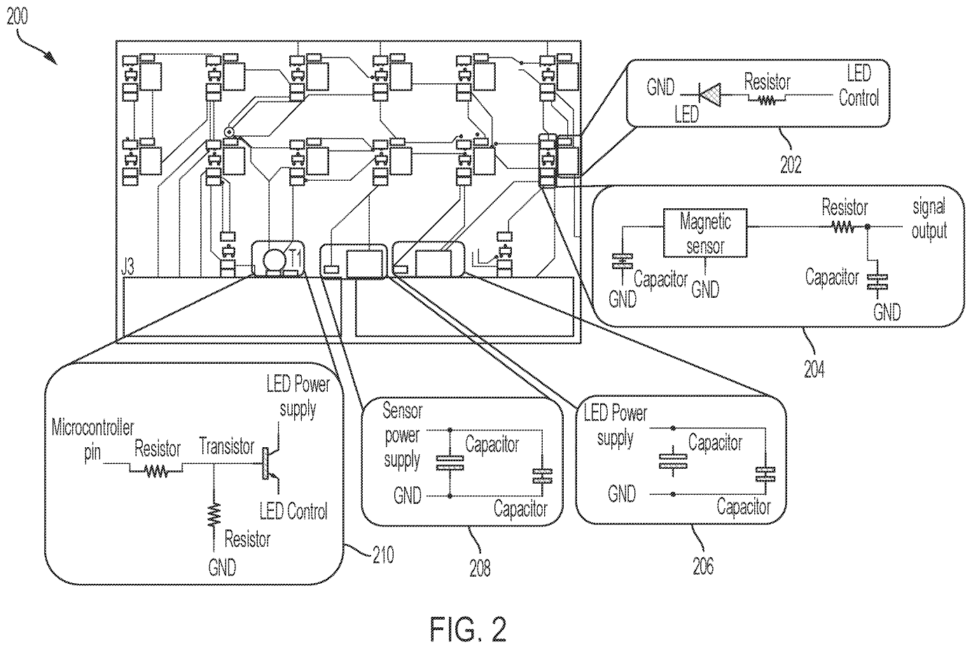

FIG. 2 is a schematic of the PCB containing magnetic sensors, LEDs, and a circuit diagram. In some examples, each magnetic sensor may be arranged on the PCB 200 along with other circuitry by locating its center about 1 mm to 3 mm away from the magnetic material toward the center of the 2-post system. The LED can also be located on the PCB next to the magnetic sensor towards the center of each tissue location. In some LED circuit 202, the LED may be connected to a resistor with appropriate resistance such as 1 kOhm. LEDs may be arranged in parallel connected to the LED power supply circuit 206 using the appropriate power supply 404 and connected to a transistor 210 that is controlled by a data acquisition card 408 or a microcontroller 406 such as Arduino. Multiple magnetic sensors circuits 204 and LED circuits 202 can be arranged on the same PCB 200 to perform medium or high throughput analysis. Possible arrangements depend on the tissue location on the tissue plate platform. To localize the PCB 200 underneath the tissue plate, a PCB plate holder 104 can be designed using Computer Aid Design software and manufactured with a 3D printer or other methods for containing the 3D tissue culture plate 126, also known as a stimulation plate that contains a plurality of wells, for example up to and including 96 wells or more for high-throughput processing, on the top of the PCB 200 with a minimum distance. In addition, a sheet of light diffuser 110 and an optical filter film 108 can also be inserted between the PCB 200 and the bottom of the tissue plate 126 to reduce the interference of excitation wavelength and provide a uniform excitation light.

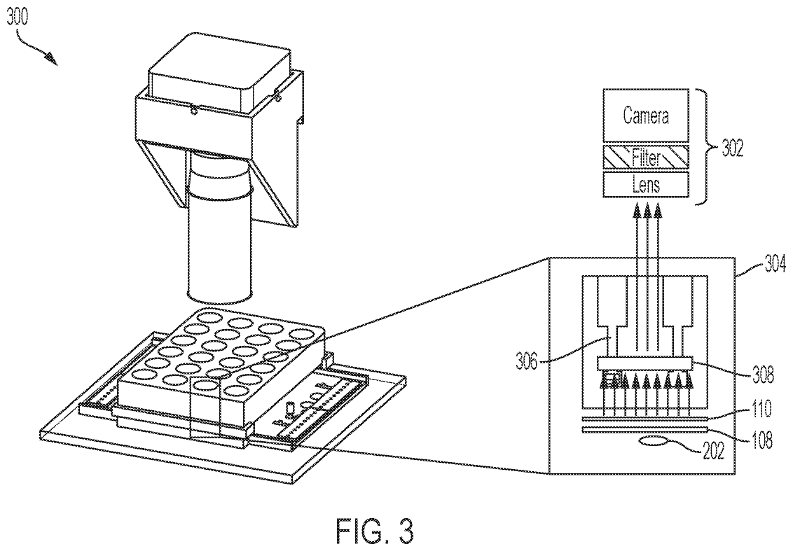

FIG. 3 is the optical monitoring unit. In the above composition, optical unit 302 may consist of camera 112, optical emission filter 116, and lens 118 mounted together for monitoring the tissue conditions and recording the calcium/voltage transient. For focusing and adjusting the camera location, a 3D motorized stage 120 such as an inexpensive fused deposition modeling 3D printer can be modified by removing the printing system (i.e., extrusion head, nozzle, and cooling fans) and replacing it with the camera mounted 114 using a 3D printed container for movement in z- and x-axes. The build platform of the 3D printer can also be modified to make a stage for the tissue plate and provide a movement in the y-axis by placing a low heat conductive material 106 (such as a 6.35 mm thick acrylic plate) between the build plate of the 3D printer and the PCB plate holder 104 to provide insulation between the PCB and the build plate. A housing module can be made by, for instance, laser cutting the acrylic plates to make the enclosure 122 surrounding the 3D motorized stage 120 with the opening at the front for inserting the tissue plate 126 or adjusting other equipment inside the housing module. Ambient temperature control can also be provided through the build platform of the 3D printer by setting the build platform temperature to, for example, 40 Celsius degrees to maintain a suitable environment for the tissue. A humidity control device 124 can also be placed inside the housing module to provide appropriate humidity.

The tissue casting method on the 2-post system can be done on medium- or high-throughput platforms such as 24- or 96-well plates by previously described techniques summarized as follows. The 2-post arrays can be obtained commercially or manually fabricated using, for example, Polydimethylsiloxane (PDMS) with one rigid post and one flexible post 306 with the embedded magnetic material at the tip, then pre-treated to enhance tissue attachment on the posts using, for instance, Polyethyleneimine and Glutaraldehyde. To cast the tissue, the reagents, including but not limited to, thrombin, fibrinogen, extracellular matrix, culture media, and cells can be mixed in the casting well to form tissue 308 around the two posts.

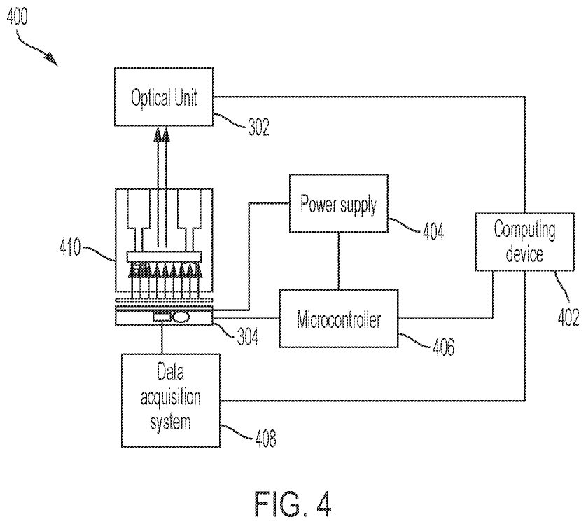

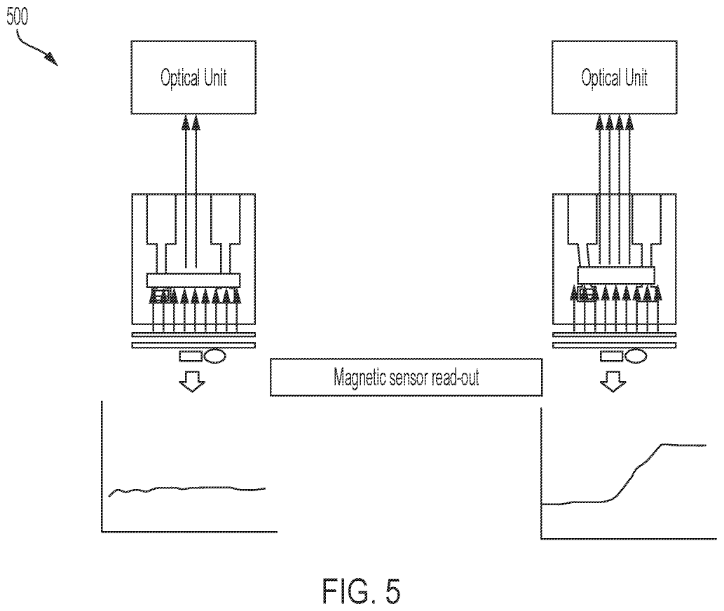

FIG. 4 is a workflow schematic of a single unit of the two-post system that is monitored using the system for simultaneous contractile force and calcium/voltage transient measurement of engineered tissue. For example, to utilize the system for simultaneous contractile force and calcium/voltage transient measurement of engineered tissue for observing muscle contraction and calcium/voltage activity, the tissue 308 is treated with the calcium-sensitive dye or the voltage-sensitive dye according to the appropriate protocol, then the tissue culture plate 126 is placed on the PCB plate holder 104 on top of the PCB 200 so that muscle tissue 308 is located above each set of magnetic sensor 204 and accompanying LED 202. The magnetic sensing output signal from each sensor can be connected to a data acquisition system 408 which then is connected to a computing device 402 such as a laptop or personal computer. The graphic user interface, such as Lab VIEW can be used to obtain and interpret the output signal as a tissue contractile force based on the previously described model. The transistor that is used to control LEDs can be connected to, for example, a 12V power supply 404 and data acquisition system 408 or a microcontroller 406 such as Arduino for turning on and off the LEDs. FIG. 5 is the illustration of simultaneous measurement of the post deflection and calcium/voltage activity. Stimulation such as electrical stimulation can be provided using a pair of electrodes for each tissue. As the electrical stimulation is provided, the muscle tissue contraction 500 can be observed through the movement of the magnetic material embedded in the flexible post 306 via the magnetic sensor on the PCB 200. Simultaneously, the calcium/voltage transient 500 in the tissue can be monitored by using the excitation light on the PCB 200 and the optical unit 302. The image processing algorithm is utilized for the analysis of calcium/voltage transient measurement associated with the contractile force derived from the magnetic sensor output signal. Together these signals are used to study and assess muscle conditions.

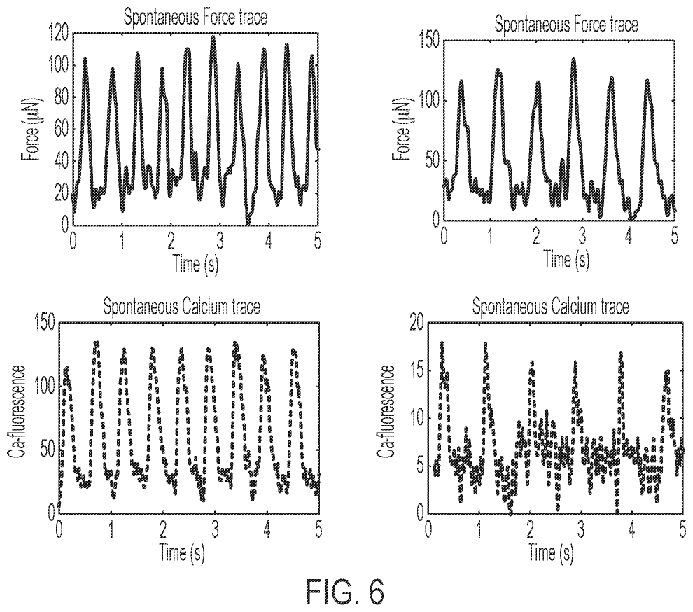

FIG. 6 is the simultaneous contractile force-calcium transient measurement preliminary data. In various examples, cardiac tissues 410 attached to posts and placed within wells of a multi-well plate may be exposed to therapeutic drugs in a variety of ways. A mixture of relevant medicine and deionized water can be used to treat cardiac tissues. Based on the final concentration, therapeutic substances can be filtered and portioned into the appropriate dilution. Some wells may act as controls in some tests, containing no medicinal substances. The spontaneous beating is recorded using 100. An example of the system output for a healthy tissue 602 and 604 shows the simultaneous measurement of the contractile force and the calcium measurement. The addition of E4031 decreases the beating rate which is confirmed by the outputs 606 and 608.

Alternatively, for the system for simultaneous contractile force and calcium/voltage transient measurement of engineered tissue, the magnetic sensor can be replaced by, for example, a high-performance 3-axis magnetometer with appropriate circuit adjustment. To improve the magnetic sensing signal output, different magnetic materials and sizes can be used. In addition, an instrumentation amplifier may be applied to enhance the signal output. Different calcium-sensitive dyes, such as CalbryteAM or Fluo-4, can be used with the appropriate LED and optical filter wavelengths. Also, different voltage-sensitive dyes can be used with the appropriate LED and optical filter wavelength.

Examples of the present invention are provided to more fully illustrate its design details. These examples may be modified into various forms. The scope of the present invention is not limited to these examples. Rather, they are provided so that the present disclosure will be more complete and fully convey the scope of the invention. In addition, dimensions such as thickness and size in the drawings are exaggerated for clarity and convenience of explanation.

### INDUSTRIAL APPLICABILITY

The present disclosure relates to a magnetic sensor and calcium/voltage transient measurement system for tissue engineering, therefore.

In summary, examples of the present disclosure provide methods, systems, and devices to provide high throughput assays that allow for simultaneously measuring the contraction and calcium/voltage transients of muscle tissue, neuromuscular tissue, vascularized cardiac tissue, or skeletal muscle tissue by using magnetic sensors for measuring contractile force and a camera for observing the calcium/voltage transients. The disclosed methods, systems, and devices provide an appropriate environment such as control temperature and humidity. For example, one imaging system that can be used is a MU313-GS camera, with a wide-angle lens or telecentric lens, FITC emission filter from Edmund Optics, and 450 nm LEDs mounted on the printed circuit board as the light source. The goal is to observe the entire 96 tissues all at once. The appropriate wavelength and optimal light intensity can be maintained for observing calcium/voltage transient instead of using a bright light as in conventional video-based motion tracking; however, in this system, magnetic sensors are used to track the motion. Also, it has been observed that increasing the light intensity and brightness could lead to photobleached and undesired background noise. The disclosed methods, systems, and devices do not use any additional pump, syringe, or fluidic device. The tissue model that is used is the two-post system that has muscle tissue cast around two posts; one is flexible, and one is rigid. The contractility is measured by magnetic sensors that are mounted on the PCB outside of the tissue platform to sense the displacement of the magnetic material embedded on the tip of the flexible post. The two-post system is fabricated by using aluminum mold obtained from milling without the need for lithography-based microfluidic fabrication. In the 2-post system in the 96-well plate platform, the initial tissue dimension is around 2 mm×4 mm. After casting, the tissue is not contained in any confined space to allow the muscle contraction function to be properly observed.

While the embodiments have been illustrated with respect to one or more implementations, alterations and/or modifications can be made to the illustrated examples without departing from the spirit and scope of the appended claims. In addition, while a particular feature of the embodiments may have been disclosed with respect to only one of several implementations, such feature may be combined with one or more other features of the other implementations as may be desired and advantageous for any given or particular function.

Furthermore, to the extent that the terms “including”, “includes”, “having”, “has”, “with”, or variants thereof are used in either the detailed description and the claims, such terms are intended to be inclusive in a manner similar to the term “comprising.” As used herein, the phrase “one or more of”, for example, A, B, and C means any of the following: either A, B, or C alone; or combinations of two, such as A and B, B and C, and A and C; or combinations of three A, B and C.

Other embodiments will be apparent to those skilled in the art from consideration of the specification and practice of the descriptions disclosed herein. It is intended that the specification and examples be considered as exemplary only, with a true scope and spirit of the embodiments being indicated by the following claims.

What is claimed is:

1. A simultaneous contractile force, calcium, or voltage measurement system, comprising:
multiple magnetic position sensors and light emitting diodes (“LEDs”) or Laser Diodes (“LDs”) arrays for monitoring tissue conditions;
an optical unit for observing tissue activity, calcium activity, voltage activity, or combinations thereof; and
a housing module for controlling temperature and carbon dioxide environment enabling 3D movement of the base or camera.

2. The simultaneous contractile force, calcium, or voltage measurement system of claim 1, wherein each of the multiple magnetic position sensors comprise analog or digital type multi-axis magnetic position sensor.

3. The simultaneous contractile force, calcium, or voltage measurement system of claim 2, wherein each of the multiple magnetic position sensors comprises a capacitor located between a voltage supply pin and a ground pin on a printed circuit board, and an output signal is connected to a bandpass filter.

4. The simultaneous contractile force, calcium, or voltage measurement system of claim 3, wherein the bandpass filter is implemented at a cutoff frequency of 21 Hz or above.

5. The simultaneous contractile force, calcium, or voltage measurement system of claim 2, wherein each of the magnetic position sensors is effective in increasing in voltage signal due to an increase detected in a magnetic field.

6. The simultaneous contractile force, calcium, or voltage measurement system of claim 1, further comprising an optical unit that comprises the LEDs or the LDs with an appropriate wavelength for providing light to excite calcium dye.

7. The simultaneous contractile force, calcium, or voltage measurement system of claim 6, further comprising a light filter film with the wavelength cutoff between the required excitation wavelength and emission wavelength and light diffusor sheet are stacked on top of the LEDs to improve the quality and uniformity of the overall excitation light.

8. The simultaneous contractile force, calcium, or voltage measurement system of claim 6, further comprising a camera that is mounted to a motorized stage, and an appropriate optical emission filter and lens are applied to monitor tissue activity.

9. The simultaneous contractile force, calcium, or voltage measurement system of claim 3, wherein each of the magnetic position sensors and LED or LD circuits are arranged together along with auxiliary circuitry that sync, filters, amplifies, or both filters and amplifies the output signal on a printed circuit board according to a location of the tissue.

10. The simultaneous contractile force, calcium, or voltage measurement system of claim 1, wherein the housing module comprises a multi motorized axis robot to precisely control camera movement for adjusting focus and to control the temperature and an enclosure to control the ambient light.

11. The simultaneous contractile force, calcium, or voltage measurement system of claim 10, wherein the multi-motorized axis robot comprises a computer-controlled actuation system.

12. The simultaneous contractile force, calcium, or voltage measurement system of claim 10, wherein the camera is mounted to one of the 2D surfaces comprised of two available axes of the robot, and the PCB is localized in the auxiliary container which allows the tissue plate to fit above and within the effective working range of magnetic position sensors; the container may be placed in between of the camera and auxiliary container for tissue conditions observation.

13. The simultaneous contractile force, calcium, or voltage measurement system of claim 1, wherein the tissue is muscle tissue, neuromuscular tissue, vascularized cardiac tissue, or skeletal muscle tissue.

14. The simultaneous contractile force, calcium, or voltage measurement system of claim 1, further comprising a stimulation plate that comprises a plurality of wells.

15. The simultaneous contractile force, calcium, or voltage measurement system of claim 14, wherein the stimulation plate comprises 24 wells, 40 wells, or 96 wells.

16. A method comprising:
culturing muscle tissue, neuromuscular tissue, vascularized cardiac tissue, or skeletal muscle tissue around a 2-post system, wherein the 2-post system comprises a flexible post that contains a magnetic portion and a rigid post;
providing stimulation such as electrical stimulation to the tissue for observing contractile function in the housing module with appropriate temperature-controlled;
detecting a change in a magnetic field as a result of a deflection of the flexible post and determining the force from the filtered signal;
detecting the calcium transient by using calcium-sensitive dye or detecting voltage transient by using voltage-sensitive dye;
providing the appropriate excitation light; and
monitoring the fluorescence signal via an optical unit.

17. The method of claim 16, wherein the stimulation comprises electrical stimulation, mechanical actuation, or optogenetic stimulation of a biological sample.
